(19) United States
(12) Patent Application Publication
(10) Pub. No.: US 2024/0409878 A1
(43) Pub. Date: Dec. 12, 2024

(54) SYSTEM AND METHOD FOR PRESERVING ALGAE

(71) Applicant: Macro Oceans Inc., San Francisco, CA (US)

(72) Inventors: Mohammad Tajparast, San Francisco, CA (US); Archana Mishra, San Francisco, CA (US); Adam Vera, San Francisco, CA (US)

(73) Assignee: Macro Oceans Inc., San Francisco, CA (US)

(21) Appl. No.: 18/740,177

(22) Filed: Jun. 11, 2024

Related U.S. Application Data
(60) Provisional application No. 63/507,708, filed on Jun. 12, 2023.

(51) Int. Cl. C12N 1/12

(52) U.S. Cl. CPC C12N 1/12

(57) ABSTRACT

A mixture can include seaweed and at least about 8% by weight, relative to a wet weight of the seaweed, alcohol. A method for preserving an algae sample can include mixing the algae sample with an alcohol, wherein a concentration of the alcohol is greater than about 5% by weight of the algae sample on a wet basis of the algae sample.

### CROSS-REFERENCE TO RELATED APPLICATIONS

This application claims the benefit of U.S. Provisional Application No. 63/507,708 filed 12 Jun. 2023, which is incorporated in its entirety by this reference.

### TECHNICAL FIELD

This invention relates generally to the algae preservation field, and more specifically to a new and useful system and method in the algae preservation field.

### BRIEF DESCRIPTION OF THE FIGURES

FIG. 1 is a flowchart representation of an example of the method; and

FIGS. 2A and 2B are schematic representations of examples of the method.

### DETAILED DESCRIPTION

The following description of the embodiments of the invention is not intended to limit the invention to these embodiments, but rather to enable any person skilled in the art to make and use this invention.

### 1. Overview

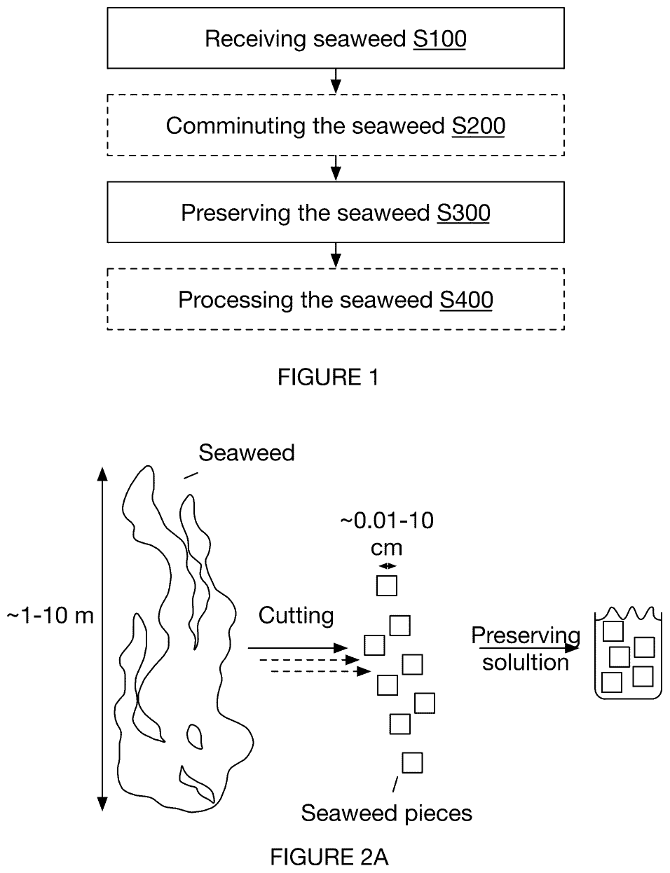

As shown in FIG. 1, the method can include receiving an algae sample, optionally processing the received algae sample, preserving the received algae sample, and optionally processing the preserved algae sample. However, the method can additionally or alternatively include any suitable steps. Examples of algae samples include (but are not limited to): red seaweed (e.g., Rhodophyta such as Aglaothamnion diaphanum, Algaothamnion priceanum, Ahnfeltia plicata, Anotrichium barbatum, Asparagopsis armata, Bornetia secundiflora, Calliblepharis ciliata, Calliblepharis jubata, Catenella caespitosa, Ceramium virgatum, Chondria coerulescens, Chondrus crispus, Corallina officinalis, Cruoria cruoriiformis, Cryptopleura ramose, Delesseria sanguinea, Dermocorynus montagnei, Dilsea carnosa, Drachiella spectabilis, Furcellaria lumbricalis, Gelidium pusillum, Gracilaria gracilis Gracilaria multipartita, Heterosiphonia plumosa, Hildenbrandia rubra, Hypoglossum hypoglossoides, Lithophyllum incrustans, Lithothamnion coralliodides, Lithothamnion glaciale, Lomentaria articulata, Mastocarpus stellatus, Membranoptera alata, Mesophyllum Lichenoides, Metacallophyllis laciniata, Odonthalia dentata, Osmundea pinnatifida, Palmaria palmata, Phycodrys rubens, Phyllophora crispa, Phymatolithon calcareum, Plocamium carilagineum, Plumeria plumosa, Polyides rotunda, Pterothamnion plumula, Rhodothamniella floridula Vertebrata fucoides, Vertebrata lanosa, Vertebrata reptabunda, Xiphosiphonia pennata, Porphyra umbilicalis, etc.), brown and/or yellow-green seaweed (e.g., xanthophyceae, phaeophyceae, ochrophyta such as Alaria esculenta, Ascophyllum nodosum, Asperococcus scaber, Bifurcaria bifurcate, Carpomitra costata, Chorda filum, Colpomenia peregrina, Cystoseira tamariscifolia, Dictyopteris polypodioides, Fucus ceranoides, Fucus distichus, Fucus serratus, Fucus spiralis, Fucus vesiculosus, Halidrys siliquosa, Halopteris filicina, Himanthalia elongate, Laminaria digitata, Laminaria hyperborea, Laminara ochroleuca, Padina pavonica, Pelvetia canaliculate, Saccharina latissimi, Saccorhiza polyschides, Sargassum muticum, Undaria pinnatifida, Zanardinia typus, nereocytis, etc.), green seaweed (e.g., streptophyta, prasinodermophhyta, zygnematophycaea, etc. such as Blidingia minima, Bryopsis plumosa, Chaetomorpha linum, cladophora liniformis, Cladophora rupestris, Codium fragile subsp. Fragile, Codium tomentosum, Derbesia marina, Lychaete battersii, Ulva intestinalis, Ulva lactuca, Ulva lunza, Urospora wormskioldii, Prasiola stipitate, etc.), stoneworts (e.g., charophyta such as Chara baltica, Chara canescens, Lamprothamnium papulosum, Tolypella nidifica, etc.), bluegreen algae (e.g., cyanobacteria), kelp, and/or any suitable plant(s).

The method (and/or a system for performing the method) preferably function to preserve the algae sample without significant degradation. For example, the macronutrients, minerals, vitamins, and/or other cellular components of the algae can be preserved without substantially degrading (e.g., with less than about 1%, 2%, 5%, 10%, 20%, etc. change in total amount of species; with less than a change of 1%, 2%, 5%, 10%, 20%, etc. change in mass of the species; without changing a biological availability of the species; without changing a biological function of the species; etc.). In a specific example, carbohydrates of the algae sample do not substantially degrade (e.g., polysaccharides do not substantially degrade or depolymerize into oligosaccharides or short-chain saccharides). In variations of this specific example, proteins and/or lipids of the algae sample may degrade (e.g., denature, oxidize, etc.) as a result of or independent of the preservation conditions. The algae sample is preferably preserved for at least a preservation time. The preservation time can be about 1 month, 2 months, 4 months, 6 months, 8 months, 12 months, 15 months, 18 months, 24 months, 36 months, 60 months, 120 months, 240 months, values or ranges therebetween, >240 months, and/or <1 month. As a specific example, the algae sample can be preserved for approximately 1 year, which can provide a technical advantage of spreading a time for processing an algae harvest over a year (where algae harvested in a given year is approximately fully processed by the time of an algae harvest in a subsequent year).

As a specific example, a method can include: receiving a seaweed sample (e.g., from a seaweed harvest), optionally rinsing the seaweed sample with fresh water, comminuting (e.g., cutting, grinding, shredding, etc.) the seaweed sample to form seaweed pieces with a size between about 500 um and about 25 cm (e.g., 0.1-2 cm, 0.5-5 cm, 1-2 cm, 2-5 cm, 1-5 cm, 1-10 cm, 0.5-20 cm, 10-50 cm, values or ranges contained therebetween, etc.), mixing the seaweed sample with a preserving solution (e.g., an ethanol solution, a sodium carbonate solution, sodium bicarbonate solution, sodium chloride solution, a basic solution such as with a pH greater than about 7, etc.), storing the seaweed sample in the preserving solution (e.g., for a storage time such as 1 day, 3 days, 1 week, 1 month, 2 months, 3 months, 6 months, 1 year, values or ranges therebetween, etc.), and optionally processing the preserved seaweed sample such as to obtain (e.g., extract) one or more product from the preserved seaweed (where the product(s) are preferably substantially the same regardless of the amount of time the seaweed sample spends in the preserving solution). However, the method can include any suitable steps.

### 2. Technical Advantages

Variants of the technology can confer one or more advantages over conventional technologies.

First, the inventors have discovered that seaweed preservation techniques used in tropical, subtropical, and/or temperate climates (e.g., in climates such as those associated with Köppen-Geiger classifications Cfa, Cwa, Cfb, Af, Am, Aw, etc.) such as found in coastal regions near the equator (e.g., along the coasts of China, Japan, Korea, etc.) are not as effective in cold or polar climates (e.g., in climates or regions associated with Köppen-Geiger classifications Dwa, Dwb, Dwc, Dwd, Dfa, Dfb, Dfc,Dfd, Dsa, Dsb, Dsc, Dsd, ET, Ef, etc.). For instance, in tropical subtropical, and/or temperate climates, seaweed can be preserved using drying (e.g., air drying, solar drying, etc.) such as by hanging or suspending the seaweed for a threshold amount of time (e.g., a time contained within the time range of 1 hour-7 days) in sunlight. When similar preservation techniques are using in cold or polar climates, the seaweed can be prone to degradation (e.g., rotting, molding, etc.) rather than drying (particularly as the seaweed harvest season typically coincides with Spring months). To overcome this deficiency, the inventors have found that treating the seaweed with a preserving solution (e.g., ethanol, saline, alkaline, etc. solution) can preserve the seaweed. These solutions have an additional benefit of requiring less time, space, and/or energy than implementing a mechanism for drying to preserve the seaweed in these climates.

Second, variants of the technology that use a gentle preservation technique can decrease an amount of seaweed product degradation resulting from the preservation. As a specific example, the use of a preserving solution (e.g., alcohol, saline solution, alkaline solution, etc.) can help delay and/or avoid degradation of seaweed products such as carbohydrates (e.g., polysaccharides such as alginic acid or salts derived therefrom, carrageenans, agar, etc.; oligosaccharides; disaccharides; monosaccharides; sugars; starches; cellulose; etc.) lipids, proteins, ash or minerals (e.g., iodine, potassium, etc.), and/or other seaweed products. For instance, over the course of a year, a polysaccharide and/or oligosaccharide length can remain substantially constant (e.g., decrease by at most 10% by mass, result in at most 10% mass of monosaccharides derived from the poly-or oligosaccharides, etc.).

Third, variants of the technology can enable ‘greener’ seaweed processing. For instance, variants of the technology can be more environmentally friendly, use less energy, require less space, and/or can otherwise facilitate green processing by not drying the seaweed before or during seaweed preservation (e.g., because seaweed drying can be energy intensive, space intensive, etc. depending on a climate). In related variants, the exclusion of drying can reduce steps during seaweed processing (whereas traditional seaweed processing pipelines would use drying to preserve the seaweed before rehydrating the dried seaweed for further processing steps). Additionally or alternatively, the exclusion of drying may facilitate preservation of seaweed products (e.g., one or more seaweed products can be degraded by drying techniques and thus exclusion of drying can lead to less degradation of these products).

Fourth, the inventors have discovered that there is a critical preservation solution concentration to achieve preservation of the algae sample. The critical preservation solution concentration can depend on the amount of time the algae sample is to be preserved for, a preservation temperature, an algae species (or other taxonomical rank such as phylum, class, order, family, genus, intermediate ranks therebetween associated with the algae), humidity, moisture content, wet mass content of the algae sample (e.g., composition of the wet mass of the algae sample), dry mass content of the algae sample (e.g., composition of the dry mass of the algae sample), and/or other suitable. For instance, to preserve an algae sample (e.g., sugar kelp, ribbon kelp, bull kelp, brown alga, etc.) for 2 months or less, a critical preservation solution for preserving an algae sample over temperature excursions between 0° F. and 140° F. could use at least about 5 wt % (e.g., 4.8% or greater) alcohol or at least about a 9 wt % (e.g., 8.75% or greater) sodium carbonate solution (e.g., on a wet mass basis relative to algae mass). As another example, to preserve an algae sample (e.g., sugar kelp, ribbon kelp, bull kelp, brown alga, etc.) for 12 months or less over temperature excursions between 0° F. and 140° F., a critical preservation solution could use at least about 8 wt % (e.g., 7. 5% or greater) alcohol (e.g., on a wet mass basis relative to algae mass). However, other critical values can be determined and/or arrived at (e.g., by controlling temperature excursions particularly high temperature range, for different types of algae, using a mixture of preserving agents to reduce the concentration of any single preserving agent, based on the amount of time to preserve the algae, etc.).

However, further advantages can be provided by the system and method disclosed herein.

### 3. Method

As shown in FIG. 1, the method can include receiving an algae sample, optionally processing the received algae sample, preserving the received algae sample, and optionally processing the preserved algae sample. However, the method can additionally or alternatively include any suitable steps.

The method preferably functions to preserve algae from degradation. Algae (as harvested) is typically majority water (e.g., between 75% and 99% by mass of the as harvested algae is water). While the exact composition depends on many factors (e.g., species, seasonal variations, age, size, growth, water composition, temperature, etc.), the dry (excluding the water) biomass of typical algae samples (e.g., the approximate dry composition) are predominantly (e.g., ≥50%) carbohydrates and/or fiber with other minority contributions from proteins (e.g., about 10-30%), lipid (e.g., 0.5-20%), ash (e.g., 10-20%, such as for a variety of metallic elements or salts derived therefrom including potential rare earth metals), pigments (e.g., chlorophyll; phycobiliproteins; carotenoids such as β-carotene, fucoxanthin, tocopherol, violaxanthin, antheraxanthin, zeaxanthin, lutein, neoxanthin, etc.; in concentrations typically less than about 1%), and/or other minority species. In particular embodiments, the method can function to preserve one or more of these dry mass components. For instance, the method can preserve the carbohydrates from substantial degradation (while not necessarily preserving proteins, lipids, pigments, etc. from degradation). Other instantiations of the method could preserve the proteins, lipids, pigments, and/or other components of the algae from substantial degradation (while not necessarily preserving the other components from degradation).

For example, the macronutrients, minerals, vitamins, and/or other cellular components of the algae can be preserved without substantially degrading (e.g., with less than about 1%, 2%, 5%, 10%, 20%, etc. change in total amount of species; with less than a change of 1%, 2%, 5%, 10%, 20%, etc. change in mass of the species; without changing a biological availability of the species; without changing a biological function of the species; etc.). The algae sample is preferably preserved for at least a preservation time. The preservation time can be about 1 month, 2 months, 4 months, 6 months, 8 months, 12 months, 15 months, 18 months, 24 months, 36 months, 60 months, 120 months, 240 months, values or ranges therebetween, >240 months, and/or <1 month. Often, a one-year preservation time scale is targeted, which can provide a technical advantage of spreading a time for processing an algae harvest over a year (where a first algae harvest is approximately fully processed, and a second algae harvest is occurring).

The method is typically performed within a threshold time of algae harvesting (e.g., within 1 minute, 5 minutes, 10 minutes, 20 minutes, 30 minutes, 1 hour, 2 hours, 4 hours, 6 hours, 8 hours, 12 hours, 24 hours, 36 hours, 48 hours, 72 hours, 96 hours, 120 hours, 144 hours, 1 week, etc.). All or portions of the method can be performed in real time (e.g., as seaweed or algae is harvested), concurrently, asynchronously, periodically, delayed, and/or at any other suitable time. All or portions of the method can be performed automatically, manually, semi-automatically, and/or otherwise performed.

The method is preferably performed at the same location as seaweed harvest. However, the method can additionally or alternatively be performed at a remote location from the seaweed harvest (e.g., at a dedicated processing plant). For example, when seaweed is harvested from an ocean vessel (e.g., boat, barge, floating platform, etc.), the seaweed can be processed on an ocean vessel (e.g., the harvesting ocean vessel, a second processing ocean vessel proximal the harvesting ocean vessel) as well. However, the method can be performed at any suitable location.

The method can be performed on a laboratory or bench scale (e.g., utilize operate on up to approximately 1 kg of algae or seaweed, utilize or operate on 1-10 g of algae or seaweed per hour, etc.), pilot scale (e.g., utilize operate on up to approximately 100 kg of algae or seaweed, utilize or operate on 1-10 kg of algae or seaweed per hour, etc.), demonstration scale (e.g., utilize operate on up to approximately 10 tons of algae or seaweed, utilize or operate on 1-10 tons of algae or seaweed per hour, etc.), commercial scale (e.g., utilize operate on up to approximately 100 kilotons of algae or seaweed, utilize or operate on 100-1000 tons of algae or seaweed per hour, etc.), and/or can be used on any suitable scale.

The method and/or steps thereof can be performed in a batch process, as a stream (or continuous) process, and/or as any suitable process.

Receiving an algae sample S100 can function to acquire an algae sample to be preserved and/or processed. The algae sample is preferably a seaweed (e.g., from the clade stramenophiles, diaphoretickes, etc.; phylum gyrista, cyanidophyta, rhodophyta, etc.; subphylum ochrophytina, rhodellophytine, metarhodophytina, eurodophytine, etc.; infraphylum chrysista, diatomista, etc.; class phaeophyceae, xanthophyceae, Chlorophyceae, trebouxiophyceae, ulvophyceae, chlorodendrophyceae, pedinophyceae, chloropicophyceae, picocystophyceae, nephroselmidophyceae, zygnematophyceae, coleochaetophyceae, Charophyceae, mamiellophyceae, pyramimonadophyceae, pamophyllophyceae, pasinodermophyceae, florideophyceae, rhodellophyceae, compsopogonophyceae, bangiophyceae, florideophyceae, etc.). However, the algae sample can additionally or alternatively include any suitable algae.

The algae sample is preferably received during (e.g., simultaneously, concurrently, contemporaneously, etc.) or shortly after (e.g., within about 1 hour of, 2 hours of, 4 hours of, 6 hours of, 8 hours of, 12 hours of, 16 hours of, 18 hours of, 20 hours of, 24 hours of, etc.) harvest. However, the algae sample can be received any suitable time after the algae is harvested (e.g., before the algae has degraded; after the algae has degraded such as in a specified manner, by a predetermined amount, etc.; etc.) and/or at any suitable time (e.g., an algae sample that has been preserved according to a first method can be received with any suitable delay relative to the preservation time of that first method such as to transition from one preserving agent to a new preserving agent).

The algae sample is preferably received at a processing plant (e.g., preserving plant). However, the algae sample can be received at any suitable location. In a specific example, the processing plant can be the same as a harvesting boat that harvests algae. In a variation of this specific example, the processing plant can be a second boat that travels with the harvesting boat, where the algae can be harvested by the harvesting boat and preserved on the preserving boat. However, the algae can be received to be processed (e.g., in S200, in S400, etc.), to be preserved (e.g., in S300), and/or can otherwise be received at any suitable location(s).

Processing the received algae sample S200 can function to prepare the algae sample (e.g., algae sample as received in S100) for preservation (e.g., as performed in S300). Processing the received algae sample can additionally or alternatively function to make the algae sample easier to work with (e.g., easier to transport, preservation techniques more efficient, preservation techniques require less reagents, etc.), and/or can otherwise function. The algae sample is preferably processed before preservation. However, the algae sample can additionally or alternatively be processed during and/or after preservation. S200 is typically performed before S300. However, S200 could be performed concurrently with and/or after S300 (e.g., a comminution mechanism can be used to mix the algae sample with the preserving solution at the same time as the algae sample is comminuted).

In variants, processing the received algae sample can include washing the algae sample S220, comminuting the algae sample S270, and/or any suitable processing steps can be performed.

Washing the algae sample S220 can function to remove dirt, microorganisms (e.g., bacteria, viruses, fungi, microbes, etc.), and/or other undesirable species (e.g., that will result in reduced preservation performance, that will degrade the algae, etc. such as enzymes, toxins, etc.) from the algae sample. The algae sample can be washed with fresh water, filtered seawater, solvent(s) (e.g., organic solvents such as alcohols, ethers, etc.), disinfectant solution(s) (e.g., air disinfectants such as bleach, glycols, propylene glycol, triethylene glycol, etc.; alcohols such as ethanol, isopropyl alcohol, methanol, etc.; aldehydes such as formaldehyde, glutaraldehyde, ortho-phthalaldehyde, etc.; oxidizing agents such as anolyte, hydrogen peroxide, accelerated hydrogen peroxide, ozone, potassium permanganate, etc.; peroxy-and/or peroxo-acids such as peroxyformic acid, peracetic acid, peroxypropionic acid, monoperoxyglutatix acid, monoperoxysuccinic acid, peroxybenzoic acid, peroxyanisic acid, chloroperbenzoic acid, monoperoxyphthalix acid, peroxymonosulfuric acid, etc.; phenolics such as phenol, o-phenylphenol, chloroxylenol, hexchlorophene, thymol, amylmetacresol, 2,4 dichlorobenzyl alcohol, etc.; quaternary ammonium compounds such as benzalkonium chloride, benzethonium chloride, etc.; chlorine such as sodium hypochlorite, calcium hypochlorite, monochloramine, chloramine-T, trichloroisocyanuric acid, chlorine dioxide, hypochlorous acid, etc.; iodine such as iodine, iodophors, etc.; acids and/or bases such as sodium hydroxide, potassium hydroxide, calcium hydroxide, magnesium hydroxide, sulfurous acid, sulfur dioxide, phosphoric acid, dodecylbenzesulfonic acid, etc.; metals such as aluminium, antimony, arsenic, barium, bismuth, boron, copper, gold, lead, mercury, nickel, silver, thallium, tin, zinc, etc.; terpenes such as thymol, pine oil, etc.; polyaminopropyl biguanide; sodium bicarbonate; etc.), and/or using any suitable washing material. The washing material typically depends on the product and/or application of the algae products to be extracted (e.g., in S400). However, the washing material can additionally or alternatively depend on the preserving solution, the preserving time, the extraction method, the algae sample (e.g., type of algae, types of pathogens or other species present in the algae sample, etc.), and/or can otherwise be selected.

In variants of the method that include washing the algae sample, the algae sample can be washed once or a plurality of times (e.g., with the same or different washing solutions).

In some variants, washing the algae sample can include non-chemically treating the algae sample (e.g., exposing the algae sample to UV radiation, heating the algae sample, cold plasma treatment, ultrasonic treatment, etc.).

Comminuting the algae sample S270 functions to reduce a size of the algae sample, which can be beneficial for increasing a surface area of the algae sample (e.g., thereby facilitating preservation of the algae sample), for improving transportability of the algae sample (e.g., by reducing a size of the algae sample, by facilitating slurry formation of the algae sample, etc.), and/or can otherwise be beneficial. Comminuting the algae sample can include cutting the algae sample, grinding the algae sample, chopping the algae sample, reaping the algae sample, shredding the algae sample, and/or other comminution techniques.

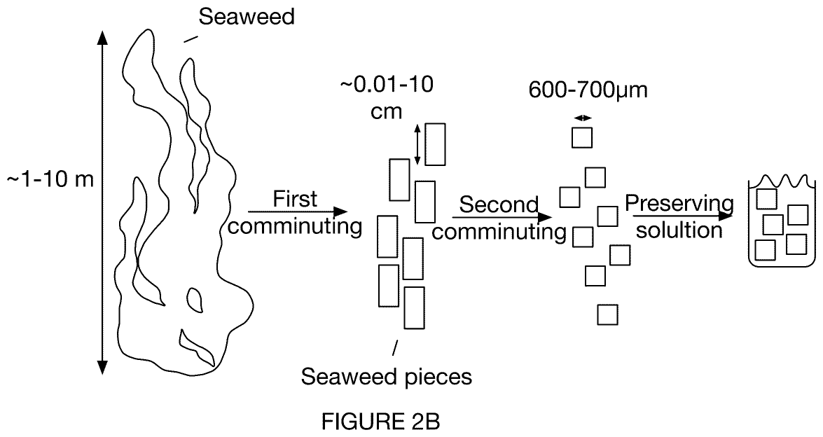

Before comminuting the algae sample, the algae are typically on the order of about 10 meters long (e.g., between about 1-100 meters, inclusive of values therebetween). However, the algae sample can be shorter than 1 meter or longer than 100 meters. After comminution, the algae sample (e.g., a length thereof, a characteristic size thereof, etc.) can be between about 100 μm and 0.5 meters (e.g., 0.1 mm, 0.15 mm, 0.2 mm, 0.4 mm, 0.5 mm, 0.7 mm, 0.8 mm, 0.9 mm, 1 mm, 2 mm, 5 mm, 10 mm, 20 mm, 50 mm, 100 mm, 200 mm, 500 mm, 0.1 in, 0.2 in, 0.5 in, 1 in, 2 in, 4 in, 6 in, 12 in, values or ranges therebetween, etc. as shown for example in FIG. 2A). However, in some variants, the algae sample can be less than about 100 μm or greater than about 0.5 m after comminution.

In one specific example, the algae sample can be comminuted to a size between about 0.5 and 10 in (e.g., 1 cm, 2 cm, 3 cm, 4 cm, 5 cm, 6 cm, 8 cm, 10 cm, 12 cm, 15 cm, 17 cm, 20 cm, 25 cm, values or ranges therebetween, etc.), which can be beneficial as this size range is easier to move and/or mix (as compared to full length algae samples) while avoiding excessive damage to the algae sample (e.g., by chopping the algae sample too finely).

In another specific example, the algae sample can be comminuted to a size between about 500 μm and 1 cm. This size range can be particularly beneficial for producing slurries of the algae sample (which can be beneficial for transfer, transport, etc. of the algae sample) and/or for avoiding excessive damage of the algae sample (e.g., by chopping the algae sample too finely).

In some variants, comminuting the algae sample can be performed in a plurality of steps (e.g., can be performed stepwise). Each of the steps can use the same and/or different comminution process (e.g., cutting, grinding, blending, chopping, etc.). For instance, as shown in FIG. 2B, a two-step comminution process can be used where after the first comminution step, the algae sample has a size between about 0.01-10 cm and after the second comminution step, the algae sample has a size between about 600-700 μm. However, the stepwise process can include any suitable number of steps and the size of the comminuted algae sample can have any suitable size.

The comminution method and/or target size after comminution can depend on the product and/or application of the algae products to be extracted (e.g., in S400), on the preserving solution, the preserving time, the extraction method, the algae sample (e.g., type of algae, types of pathogens or other species present in the algae sample, etc.), and/or can otherwise be selected.

Preserving the received algae sample S300 preferably functions to extend a lifetime of the algae sample without significant degradation of the algae sample and/or products thereof or derived therefrom. S300 can preserve an as received algae sample (e.g., from S100, fresh harvested algae sample, etc.), a processed algae sample (e.g., from S200, an algae sample that was received after one or more processing steps such as in S200 or S400 has been performed, etc.), a previously preserved algae sample (e.g., preserved according to S300, preserved in a manner different from S300, dried algae sample, etc.), and/or any suitable algae sample.

The algae sample is preferably preserved in a food-safe manner (e.g., by using nondenatured or undenatured preserving agents). However, in some embodiments, the algae sample can be preserved in a non-food safe manner (e.g., can render, result in, etc. algae that is not food safe, that is not generally recognized as safe to consume, etc. such as using a preserving agent that is not generally recognized as safe for human consumption, a denatured preserving agent, etc.).

The algae sample is typically not agitated (e.g., mixed) during (e.g., throughout) preservation (e.g., to simplify the preservation process). Alternatively stated, the algae sample is preferably mixed at most only once (e.g., at the beginning of S300 to homogenize the preserving solution and algae sample) and remains unmixed thereafter. However, the algae sample can be agitated during preservation (e.g., according to a schedule, at predetermined times, at a predetermined frequency, etc.).

The algae sample is typically preserved at approximately room temperature (e.g., an ambient temperature of an environment the algae sample is stored at). For instance, the algae sample can be preserved at a temperature between about 50° F. to 110° F. However, the algae sample can be stored at an elevated temperature (e.g., >110° F.), at a refrigeration temperature (e.g., 30-50° F.), freezing temperature (e.g., <about 30° F.), and/or at any suitable temperature. In some variants, the preserved algae sample can undergo temperature excursions as cold as about 0° F. and as high as 140° F. However, additionally or alternatively, the temperature can be controlled to within a target temperature range (e.g., a preservation temperature, a preservation temperature excursion range, etc.).

The algae sample is typically not fully sealed during preservation (e.g., evaporation of solvent, preservative, etc. can occur such as because the sample is loosely covered). Not requiring fully sealing the algae sample can be beneficial for rapidly preserving the algae sample at the time of harvest, for simplifying the equipment necessary for preserving the algae sample, and/or can otherwise be beneficial. For example, the algae sample can be preserved in a container where a lid is placed on the container to close the container but where the lid need not form a seal (e.g., is not a hermetic lid). However, the algae sample can be fully sealed (e.g., hermetically sealed), open (e.g., uncovered such as where additional solvent, preserving solution, reagent, etc. is added), and/or have any suitable interaction with the environment.

The algae sample is preferably preserved chemically (e.g., using a preserving solution). However, the algae sample can be preserved physically (e.g., by removing any potential degradation mechanisms such as pathogens, enzymes, etc. and sealing the algae sample to prevent additional degradation mechanisms such as light, pathogens, heat, etc. from reaching the algae) and/or using any suitable mechanism.

In various variants, the preserving solution can be basic (e.g., have a pH greater than about 7), acidic (e.g., have a pH less than about 7), and/or approximately neutral (e.g., have a pH between about 6.5 and 7.5).

In some embodiments, the preserving solution concentration (e.g., reagent concentration, relative concentration to the amount of algae sample, etc.) can be particularly important to control. When the preserving solution concentration exceeds a threshold concentration, the preserving solution can result in degradation of the algae sample and/or products thereof or derived therefrom, extraction of one or more species from the algae sample (where desirable extracted species can become difficult to separate from other extracted species extracted by the preserving solution rather than a preferred extraction process), can be expensive, and/or can otherwise be undesirable. When the preserving solution concentration is less than a threshold concentration, the preserving solution can be insufficiently effective at preserving the algae sample (e.g., can preserve the seaweed sample for less than a target preservation time, not result in preservation of the algae sample, etc.). The threshold preserving solution concentration (e.g., maximum concentration, minimum concentration, concentration range, etc.) can depend on the algae sample (e.g., type of seaweed, seaweed composition such as mineral composition within the seaweed, algae surface area, etc.), the preserving solution (e.g., chemical preserving species), temperature, preserving time (e.g., target preservation time), preserving algae products (or product derivatives) to preserve, humidity, ambient light, weather, and/or can otherwise depend on or be independent of any suitable properties.

As an illustrative example, an algae sample concentration (e.g., mass fraction, volume fraction, etc. of algae, seaweed, etc. to solvent) can be between about 0.01% and 1000% (e.g., 0.05%, 0.1%, 0.48%, 0.5%, 1%, 2.5%, 5%, 6%, 7%, 7.5%, 8%, 8.5%, 9%, 9.5%, 9.9%, 10%, 15%, 20%, 30%, 33.3%, 35%, 37%, 40%, 47%, 50%, 64%, 66.7%, 75%, 80%, 85%, 90%, 92%, 95%, 100%, 150%, 200%, 250%, 300%, 500%, 750%, 1000%, etc. where the percentage can refer to a w/w such as g_{algae}/g_{all-other-reagents}, g_{algae}/g_{specific-reagent}, etc.; w/v such as g_{algae}/ml_{all-other-reagents}, g_{algae}/ml_{specific-reagent(s)}, etc.; etc.). Alternatively phrased, the concentration of the preserving agents can be between 1000% and 0.01% (e.g., 0.05%, 0.1%, 0.48%, 0.5%, 1%, 2.5%, 5%, 6%, 7%, 7.5%, 8%, 8.5%, 9%, 9.5%, 9.9%, 10%, 15%, 20%, 30%, 33.3%, 35%, 37%, 40%, 47%, 50%, 64%, 66.7%, 75%, 80%, 85%, 90%, 92%, 95%, 100%, 150%, 200%, 250%, 300%, 500%, 750%, 1000%, etc. where the percentage can refer to a w/w such as g_{all-other-reagents}/g_{algae}, g_{specific-reagent}/g_{algae}, g_{all-other-reagents}/g_{algae-dry-biomass}, g_{specific-reagent}/g_{algae-dry-biomass}, g_{all-other-reagents}/g_{algae-wet-biomass}, g_{specific-reagent}/g_{algae-wet-biomass} etc.; v/w such as ml_{all-other-reagents}/g_{algae}, ml_{specific-reagent(s)}/g_{algae}, ml_{all-other-reagents}/g_{algae-dry-biomass}, ml_{specific-reagent(s)}/g_{algae-dry-biomass}, ml_{all-other-reagents}/g_{algae-wet-biomass}, ml_{specific-reagent(s)}/g_{algae-wet-biomass}, etc.; etc.). However, the algae sample concentration can be less than 0.01% or greater than 1000%.

In variants, the preserving solution can be a solvent (e.g., pure solvent, solvent mixture, etc.), can include one or more reagents (e.g., solutes, salts, acids, bases, soaps, excipients, etc.), combinations thereof, and/or can include any suitable constituents. Exemplary solvents include: water (e.g., purified water, distilled water, tap water, heavy water, etc.), seawater (e.g., filtered seawater such as filtered to remove microbes, pathogens, etc.), alcohol (e.g., methanol, ethanol, isopropanol, n-propanol, butanol, pentanol, hexanol, phenol, cyclohexanol, 2-methylpropan-1-ol, tert-amyl alcohol, allyl alcohol, geraniol, propargyl alcohol, menthol, ethylene glycol, etc.), ether (e.g., dimethyl ether, diethyl ether, methyl ethyl ether, etc.), aldehydes (e.g., formaldehyde, acetaldehyde, etc.), ketones (e.g., acetone, acetophenone, butanone, cyclopentanone, ethyl isopropyl ketone, etc.), organosulfur compounds (e.g., thiols, thioesters, sulfoxides, etc.), organonitrogen compounds (e.g., amines, amides, etc.), organophosphorous compounds (e.g., phosphate esters, phosphate amides, phosphonic acids, phosphinic acids, etc.), and/or can include any suitable solvent(s).

In variants where the preserving solution includes a mixture of solvents (or a plurality of solvents), the mixture is typically formed as a preserving reagent prior to adding the mixture to the algae sample. However, the preserving reagent can be formed while mixing the preserving reagents with the algae sample. For instance, alcohol and water are typically mixed in between a 1:5 and 5:1 ratio (e.g., volume ratio, mass ratio, mole ratio, etc.) where the mixture (e.g., an alcohol solution with concentration between 16.7% and 83.3% such as 20%, 25%, 30%, 33%, 40%, 44%, 45%, 50%, 60%, 66.7%, 70%, 72%, 75%, 80%, etc. by weight, by volume, by stoichiometry, etc.) can be formed prior to and/or during addition of the preserving agents to the algae sample. Similar considerations can apply when reagents include a plurality of different phases (e.g., a salt and a solvent).

Exemplary reagents can include: halides (e.g., fluorides such as lithium fluoride, sodium fluoride, potassium fluoride, rubidium fluoride, cesium fluoride, ammonium fluoride, etc.; chlorides such as lithium chloride, sodium chloride, potassium chloride, rubidium chloride, cesium chloride, ammonium chloride, etc.; bromides such as lithium bromide, sodium bromide, potassium bromide, rubidium bromide, cesium bromide, ammonium bromide, etc.; iodides such as lithium iodide, sodium iodide, potassium iodide, rubidium iodide, cesium iodide, ammonium iodide, etc.; etc.), hydroxides (e.g., alkali metal hydroxides such as lithium hydroxide, sodium hydroxide, potassium hydroxide, rubidium hydroxide, cesium hydroxide, etc.; alkaline earth metal hydroxides such as beryllium hydroxide, magnesium hydroxide, calcium hydroxide, strontium hydroxide, barium hydroxide, etc.; boron group hydroxides such as boric acid, aluminium hydroxide, gallium hydroxide, indium hydroxide, thallium hydroxide, etc.; transition and/or post-transition metal hydroxides such as manganese hydroxide, iron hydroxide, cobalt hydroxide, nickel hydroxide, copper hydroxide, zinc hydroxide, ruthenium hydroxide, rhenium hydroxide, iridium hydroxide, etc.; ammonium hydroxide; etc.), carbonates (e.g., alkali metal carbonates such as lithium carbonate, sodium carbonate, potassium carbonate, rubidium carbonate, cesium carbonate, etc.; alkaline earth metal carbonates such as beryllium carbonate, magnesium carbonate, calcium carbonate, strontium carbonate, barium carbonate, etc.; boron group carbonates such as borate carbonate, aluminium carbonate, gallium carbonate, indium carbonate, thallium carbonate, etc.; transition and/or post-transition metal carbonates such as manganese carbonate, iron carbonate, cobalt carbonate, nickel carbonate, copper carbonate, zinc carbonate, silver carbonate, palladium carbonate, cadmium carbonate, lead carbonate, etc.; ammonium carbonate; alkali metal bicarbonates such as lithium bicarbonate, sodium bicarbonate, potassium bicarbonate, rubidium bicarbonate, cesium bicarbonate, etc.; alkaline earth metal bicarbonates such as beryllium bicarbonate, magnesium bicarbonate, calcium bicarbonate, strontium bicarbonate, barium bicarbonate, etc.; boron group bicarbonates such as borate bicarbonate, aluminium bicarbonate, gallium bicarbonate, indium bicarbonate, thallium bicarbonate, etc.; transition and/or post-transition metal bicarbonates such as manganese bicarbonate, iron bicarbonate, cobalt bicarbonate, nickel bicarbonate, copper bicarbonate, zinc bicarbonate, silver bicarbonate, palladium bicarbonate, cadmium bicarbonate, lead bicarbonate, etc.; ammonium bicarbonate; carbonated water; carbon dioxide; etc.), weak bases (e.g., alanine, ammonia, methylamine, pyridine, aniline, hydrazine, etc.), phosphates (e.g., alkali metal phosphates such as lithium phosphate, sodium phosphate, potassium phosphate, rubidium phosphate, cesium phosphate, etc.; alkaline earth metal phosphates such as beryllium phosphate, magnesium phosphate, calcium phosphate, strontium phosphate, barium phosphate, etc.; boron group phosphates such as borate phosphate, aluminium phosphate, gallium phosphate, indium phosphate, thallium phosphate, etc.; transition and/or post-transition metal phosphates such as manganese phosphate, iron phosphate, cobalt phosphate, nickel phosphate, copper phosphate, zinc phosphate, silver phosphate, palladium phosphate, cadmium phosphate, lead phosphate, etc.; ammonium phosphate; phosphoric acid; etc.), citrates (e.g., alkali metal citrates such as lithium citrate, sodium citrate, potassium citrate, rubidium citrate, cesium citrate, etc.; alkaline earth metal citrates such as beryllium citrate, magnesium citrate, calcium citrate, strontium citrate, barium citrate, etc.; boron group citrates such as borate citrate, aluminium citrate, gallium citrate, indium citrate, thallium citrate, etc.; transition and/or post-transition metal citrates such as manganese citrate, iron citrate, cobalt citrate, nickel citrate, copper citrate, zinc citrate, silver citrate, palladium citrate, cadmium citrate, lead citrate, etc.; ammonium citrate; citric acid; etc.), sorbates (e.g., alkali metal sorbates such as lithium sorbate, sodium sorbate, potassium sorbate, rubidium sorbate, cesium sorbate, etc.; alkaline earth metal sorbates such as beryllium sorbate, magnesium sorbate, calcium sorbate, strontium sorbate, barium sorbate, etc.; boron group sorbates such as borate sorbate, aluminium sorbate, gallium sorbate, indium sorbate, thallium sorbate, etc.; transition and/or post-transition metal sorbates such as manganese sorbate, iron sorbate, cobalt sorbate, nickel sorbate, copper sorbate, zinc sorbate, silver sorbate, palladium sorbate, cadmium sorbate, lead sorbate, etc.; ammonium sorbate; sorbic acid; etc.), benzoates (e.g., alkali metal benzoates such as lithium benzoate, sodium benzoate, potassium benzoate, rubidium benzoate, cesium benzoate, etc.; alkaline earth metal benzoates such as beryllium benzoate, magnesium benzoate, calcium benzoate, strontium benzoate, barium benzoate, etc.; boron group benzoates such as borate benzoate, aluminium benzoate, gallium benzoate, indium benzoate, thallium benzoate, etc.; transition and/or post-transition metal benzoates such as manganese benzoate, iron benzoate, cobalt benzoate, nickel benzoate, copper benzoate, zinc benzoate, silver benzoate, palladium benzoate, cadmium benzoate, lead benzoate, etc.; ammonium benzoate; benzoic acid; etc.), sulfites (e.g., alkali metal sulfites such as lithium sulfite, sodium sulfite, potassium sulfite, rubidium sulfite, cesium sulfite, etc.; alkaline earth metal sulfites such as beryllium sulfite, magnesium sulfite, calcium sulfite, strontium sulfite, barium sulfite, etc.; boron group sulfites such as borate sulfite, aluminium sulfite, gallium sulfite, indium sulfite, thallium sulfite, etc.; transition and/or post-transition metal sulfites such as manganese sulfite, iron sulfite, cobalt sulfite, nickel sulfite, copper sulfite, zinc sulfite, silver sulfite, palladium sulfite, cadmium sulfite, lead sulfite, etc.; ammonium sulfite; sulfur dioxide; etc.), nitrites (e.g., alkali metal nitrites such as lithium nitrite, sodium nitrite, potassium nitrite, rubidium nitrite, cesium nitrite, etc.; alkaline earth metal nitrites such as beryllium nitrite, magnesium nitrite, calcium nitrite, strontium nitrite, barium nitrite, etc.; boron group nitrites such as borate nitrite, aluminium nitrite, gallium nitrite, indium nitrite, thallium nitrite, etc.; transition and/or post-transition metal nitrites such as manganese nitrite, iron nitrite, cobalt nitrite, nickel nitrite, copper nitrite, zinc nitrite, silver nitrite, palladium nitrite, cadmium nitrite, lead nitrite, etc.; ammonium nitrite; etc.), nitrates (e.g., alkali metal nitrates such as lithium nitrate, sodium nitrate, potassium nitrate, rubidium nitrate, cesium nitrate, etc.; alkaline earth metal nitrates such as beryllium nitrate, magnesium nitrate, calcium nitrate, strontium nitrate, barium nitrate, etc.; boron group nitrates such as borate nitrate, aluminium nitrate, gallium nitrate, indium nitrate, thallium nitrate, etc.; transition and/or post-transition metal nitrates such as manganese nitrate, iron nitrate, cobalt nitrate, nickel nitrate, copper nitrate, zinc nitrate, silver nitrate, palladium nitrate, cadmium nitrate, lead nitrate, etc.; ammonium nitrate; etc.), lactates (e.g., alkali metal lactates such as lithium lactate, sodium lactate, potassium lactate, rubidium lactate, cesium lactate, etc.; alkaline earth metal lactates such as beryllium lactate, magnesium lactate, calcium lactate, strontium lactate, barium lactate, etc.; boron group lactates such as borate lactate, aluminium lactate, gallium lactate, indium lactate, thallium lactate, etc.; transition and/or post-transition metal lactates such as manganese lactate, iron lactate, cobalt lactate, nickel lactate, copper lactate, zinc lactate, silver lactate, palladium lactate, cadmium lactate, lead lactate, etc.; ammonium lactate; lactic acid; etc.), propionates (e.g., alkali metal propionates such as lithium propionate, sodium propionate, potassium propionate, rubidium propionate, cesium propionate, etc.; alkaline earth metal propionates such as beryllium propionate, magnesium propionate, calcium propionate, strontium propionate, barium propionate, etc.; boron group propionates such as borate propionate, aluminium propionate, gallium propionate, indium propionate, thallium propionate, etc.; transition and/or post-transition metal propionates such as manganese propionate, iron propionate, cobalt propionate, nickel propionate, copper propionate, zinc propionate, silver propionate, palladium propionate, cadmium propionate, lead propionate, etc.; ammonium propionate; propionic acid; etc.), ascorbates (e.g., alkali metal ascorbates such as lithium ascorbate, sodium ascorbate, potassium ascorbate, rubidium ascorbate, cesium ascorbate, etc.; alkaline earth metal ascorbates such as beryllium ascorbate, magnesium ascorbate, calcium ascorbate, strontium ascorbate, barium ascorbate, etc.; boron group ascorbates such as borate ascorbate, aluminium ascorbate, gallium ascorbate, indium ascorbate, thallium ascorbate, etc.; transition and/or post-transition metal ascorbates such as manganese ascorbate, iron ascorbate, cobalt ascorbate, nickel ascorbate, copper ascorbate, zinc ascorbate, silver ascorbate, palladium ascorbate, cadmium ascorbate, lead ascorbate, etc.; ammonium ascorbate; ascorbic acid; etc.), gallates (e.g., alkali metal gallates such as lithium gallate, sodium gallate, potassium gallate, rubidium gallate, cesium gallate, etc.; alkaline earth metal gallates such as beryllium gallate, magnesium gallate, calcium gallate, strontium gallate, barium gallate, etc.; boron group gallates such as borate gallate, aluminium gallate, gallium gallate, indium gallate, thallium gallate, etc.; transition and/or post-transition metal gallates such as manganese gallate, iron gallate, cobalt gallate, nickel gallate, copper gallate, zinc gallate, silver gallate, palladium gallate, cadmium gallate, lead gallate, etc.; ammonium gallate; gallic acid; etc.), edetates (e.g., alkali metal edetates such as lithium edetate, sodium edetate, potassium edetate, rubidium edetate, cesium edetate, etc.; alkaline earth metal edetates such as beryllium edetate, magnesium edetate, calcium edetate, strontium edetate, barium edetate, sodium calcium edetate, etc.; boron group edetates such as borate edetate, aluminium edetate, gallium edetate, indium edetate, thallium edetate, etc.; transition and/or post-transition metal edetates such as manganese edetate, iron edetate, cobalt edetate, nickel edetate, copper edetate, zinc edetate, silver edetate, palladium edetate, cadmium edetate, lead edetate, etc.; ammonium edetate; edetic acid; etc.), phenols (e.g., butylated hydroxytoluene, butylated hydroxyanisole, etc.), tocopherols and/or tocotrienols (e.g., α-tocopherol, β-tocopherol, [symbol] -tocopherol, δ-tocopherol, α-tocotrienol, β-tocotrienol, [symbol] -tocotrienol, δ-tocotrienol, etc.), isothiazolinones (e.g., methylchloroisothiazolinone, methylisothiazolinone, benzisothiazolinone, octylisothiazolinone, dichlorooctylisothiazolinone, butylbenzisothiazolinone, etc.), formaldehyde releaser (e.g., hydantoins such as 1,3-bis(hydroxymethyl)-5,5-dimethylimidazolidine-2,4-dione; quaternium-15;(ethylenedioxy)dimethanol (EDDM); (benzyloxy)methanol; 2,2′,2-(hexahydro-1,3-5-triazine-1,3,5-triyl-)triethanol; tetramethylolacetylenediurea; 3,3′-methylenebis[5-methyloxazolidine]; imidazolidinyl urea; diazolidinyl urea; tris(hydroxymethyl) nitromethane; tris(n-hydroxyethyl) hexahydrotriazine; sodium hydroxymethylglycinate; etc.), and/or any suitable reagent(s) (e.g., preservative, anti-oxidant, stabilizer, etc.) can be used.

A preserving solution reagent can be formed prior to mixing with the algae sample and/or during mixing with the algae solution. For instance, a salt solution (e.g., a reagent solution) could be formed to a target concentration prior to adding the salt solution to the algae sample. The amount of reagent solution (and/or particular preserving agent) added to the algae sample can depend on the concentration of the algae sample, the quantity of algae sample to be preserved, the water content of the algae sample (e.g., actual measured water content, estimated water content based on species (or other categorization) of the algae sample, etc. which is typically between about 80 and 97% by weight of the algae sample and typically does not substantially vary for a given algae sample even across seasons), preservations time (e.g., target amount of time to preserve the algae sample), and/or can depend on any suitable property(s).

As a first specific example, alcohol (e.g., methanol, ethanol, isopropanol, etc.) can be used as the preserving solution. In this specific example, the alcohol is preferably included at a concentration that is between about 5% and 50% (e.g., weight percent, volume percent, etc. such as about 5% w/w alcohol to wet biomass solution, about 5% v/w alcohol to wet biomass solution, about 6% w/w alcohol to wet biomass solution, about 6% v/w alcohol to wet biomass solution, about 7% w/w alcohol to wet biomass solution, about 7% v/w alcohol to wet biomass solution, about 8% w/w alcohol to wet biomass solution, about 8% v/w alcohol to wet biomass solution, about 9% w/w alcohol to wet biomass solution, about 9% v/w alcohol to wet biomass solution, about 10% w/w alcohol to wet biomass solution, about 10% v/w alcohol to wet biomass solution, about 12% w/w alcohol to wet biomass solution, about 12% v/w alcohol to wet biomass solution, about 16% w/w alcohol to wet biomass solution, about 16% v/w alcohol to wet biomass solution, about 28% w/w alcohol to wet biomass solution, about 28% v/w alcohol to wet biomass solution, about 40% w/w alcohol to wet biomass solution, about 40% v/w alcohol to wet biomass solution, etc.). In a variation of this specific example that used less than 8% alcohol (e.g., 1%, 3%, 5%), the preserving solution did not preserve the algae (e.g., for the target preserving duration; for instance, instead of achieving about 1 year of preservation a 5% alcohol solution achieved a preservation duration of about 2 months before product degradation, mold growth, etc. was detected). However, concentrations less than 5% alcohol may be suitable (e.g., using different algae concentration, using a solvent system, using a sealed preservation system, for shorter target preserving durations, etc.).

In a second specific example, sodium carbonate (and/or any combination of alkali metal carbonates or other soluble carbonates) can be used as the reagent. In the second specific example, the carbonate is preferably included at a concentration that is between about 9% and 20% (e.g., weight percent, volume percent, etc. such as about 9% w/w carbonate to biomass solution, about 9% v/w carbonate to biomass solution, about 11% w/w carbonate to biomass solution, about 11% v/w carbonate to biomass solution, about 13% w/w carbonate to biomass solution, about 13% v/w carbonate to biomass solution, about 15% w/w carbonate to biomass solution, about 15% v/w carbonate to biomass solution, etc.). In a variation of the second specific example, bicarbonate (e.g., sodium bicarbonate) can be used instead of and/or in addition to carbonate. In a variation of this specific example that used less than 9% carbonate (e.g., 5%), the preserving solution did not preserve the algae (e.g., for the target preserving duration). However, concentrations less than 9% (or greater than 20%) carbonate may be suitable (e.g., using different algae concentration, using a solvent system, using a sealed preservation system, by combining with an alcohol, etc.).

In a third specific example, salt (e.g., sodium chloride, potassium chloride, magnesium chloride, etc.) can be used as the preserving reagent. In the third specific example, the salt is preferably included at a concentration that is between about 6% and 50% (e.g., weight percent, volume percent, etc. such as about 6% w/w salt to biomass solution, about 6% v/w salt to biomass solution, about 12% w/w salt to biomass solution, about 12% v/w salt to biomass solution, about 27% w/w salt to biomass solution, about 27% v/w salt to biomass solution, about 42% w/w salt to biomass solution, about 42% v/w salt to biomass solution, etc.). In a variation of this specific example that used less than 6% salt (e.g., 5%), the preserving solution did not preserve the algae (e.g., for the target preserving duration). However, concentrations less than 6% (or greater than 50%) salt may be suitable (e.g., using different algae concentration, using a solvent system, using a sealed preservation system, using the salt in combination with alcohol or other preserving solvent(s), by modifying a pH of the solution, etc.).

In a variation of the third specific example, seawater (particularly seawater that is filtered to remove microbes, pathogens, etc.) can be used to form the algae solution. As minerals in seawater differ annually, this variation may only work in specific conditions (e.g., for particularly mineral concentrations, particularly mineral concentrations in a given algae sample, for particular algae, etc.).

In a fourth specific example, benzoates (e.g., sodium benzoate) can be used as the preserving reagent. In the fourth specific example, the benzoate is preferably included at a concentration that is between about 0.5% and 5% (e.g., weight percent, volume percent, etc. such as about 0.5% w/w benzoate to biomass solution, about 0.5% v/w benzoate to biomass solution, about 1% w/w benzoate to biomass solution, about 1% v/w benzoate to biomass solution, about 2% w/w benzoate to biomass solution, about 2% v/w benzoate to biomass solution, about 4% w/w benzoate to biomass solution, about 4% v/w benzoate to biomass solution, etc.). In a variation of this specific example that used less than 0.5% benzoate (e.g., 0.1%), the preserving solution did not preserve the algae (e.g., for the target preserving duration). However, concentrations less than 0.5% (or greater than 5%) benzoate may be suitable (e.g., using different algae concentration, using a solvent system, using a sealed preservation system, etc.).

In a fifth specific example, two or more of the preceding first through fourth specific examples of preserving reagents can be combined and/or used in concert. In variations, a plurality of preserving agents can be used concurrently (e.g., mixed to form the preserving solution). In other variations, the plurality of preserving agents can be used sequentially (e.g., a first preserving solution is used for a first period of time followed by a second preserving solution and so on). However, a plurality of preserving solutions can be used in any manner.

As an illustrative example, for a sugar kelp sample that is assumed to have a moisture content of about 90%, for every 1 kg of sugar kelp that is added, about 80 g of alcohol (e.g., ethanol, methanol, isopropyl alcohol, etc.) can be added to the sugar kelp to preserve the sugar kelp for at least one year (e.g., resulting in an approximately 8 wt % alcohol relative to the wet biomass of the sugar kelp). In a variation of this illustrative example, about 100 g of water can be added to the sugar kelp per kg of sugar kelp, where the inclusion of additional water can be beneficial for mixing and/or transporting the sugar kelp. Similar considerations can be applied for other preserving agents.

Preserving the algae sample preferably does not include drying the algae sample. These variants can be particularly beneficial for some products and/or derived products as the dehydrated algae would have to be rehydrated to obtain the materials and/or because dehydration can degrade or change the materials. However, in some variants, preserving the algae sample can include drying the algae sample (e.g., partially or fully dehydrating the algae sample), which can be beneficial for reducing a weight and/or volume of the algae sample (e.g., for storage, for shipping, etc.).

Preserving the algae sample preferably does not include freezing the algae sample. These variants can be particularly beneficial for reducing capital costs (as refrigeration units can be expensive depending on a quantity of algae sample to be preserved). However, preserving the algae sample can include freezing the algae sample (e.g., incidentally when an environment temperature happens to fall below a freezing point of the algae sample, intentionally, etc.).

Preserving the algae sample preferably does not include fermenting the algae sample (e.g., silage-based preservation). These variants can be particularly beneficial as fermentation generally results in degradation of carbohydrates into alcohol(s) (and degradation of carbohydrates is undesirable in most embodiments of the technology). However, silage-based preservation can be used (e.g., a first algae sample can be preserved via silage where alcohol generated by the silage preservation can be used to preserve other algae sample(s)).

Processing the preserved algae S400 sample preferably functions to obtain (e.g., extract) one or more product from the algae sample. S400 is preferably performed on For instance, processing the preserved algae sample can include extracting minerals, nutrient, macromolecules, and/or other suitable species form the algae sample. Processing the algae sample can include washing the algae sample (e.g., to remove residual preservative solution, reagents, etc.), acid and/or base extraction, electrochemical extraction, magnetic extraction, leeching, crushing, comminution, and/or other suitable steps to obtain one or more species (e.g., chemical species) from the algae sample. For instance, laminarin, fucoidan, mannitol, alginic acid or alginates derived therefrom, ulvan, glucan, carageenans, agar and/or other suitable polysaccharides and/or carbohydrates can be extracted from the algae sample, However, the algae sample can be processed without washing the algae sample (e.g., where the preserving solution can be beneficial for, enhance, etc. the processing).

In a first illustrative example, a method for preserving an algae sample can comprise mixing the algae sample with an alcohol, wherein a concentration of the alcohol is at least about 8% by weight of the algae sample on a wet basis of the algae sample.

In a second illustrative example, the method of the first illustrative example can further comprise comminuting the algae sample prior to mixing the algae sample with the alcohol.

In a third illustrative example, the method of the second illustrative example, wherein after comminuting the algae sample, an average size of algae of the algae sample is between about 500 μm and about 10 cm (e.g., 0.5-10 cm, 1-5 cm, 2-5 cm, 3-5 cm, 2-4 cm, 0.5-1 mm, 0.6-0.8 mm, 0.5-50 mm, etc.).

In a fourth illustrative example, the method of the second illustrative example, wherein comminuting the algae sample comprises cutting, grinding, high pressure homogenization, or shredding the algae sample.

In a fifth illustrative example, the method of any of the first through fourth illustrative examples can further comprise storing the algae sample for a duration of time up to (or exceeding) about one year.

In a sixth illustrative example, the method of the fifth illustrative example, wherein storing the algae sample comprises storing the algae sample at a temperature between −4° C. and 50° C. (e.g., wherein temperature excursions during storage do not exceed −4° C. nor 50° C.).

In a seventh illustrative example, the method of any of the fifth or sixth illustrative examples, wherein storing the algae sample comprises storing the algae sample in an unsealed container.

In an eigth illustrative example, the method of any of the fifth through seventh illustrative examples, wherein after the duration of time, a carbohydrate composition of the algae sample does not substantially change.

In a ninth illustrative example, the method of eigth illustrative examples, wherein after the duration of time, a polysaccharide concentration (and composition) of the algae sample does not substantially change (e.g., polysaccharides do not denature or depolymerize, polysaccharides remain in solid phase, polysaccharides transfer to liquid phase but do not degrade, total polysaccharide concentration between liquid phase and solid phase change by less than 5%, etc.). In some variations, one or more polysaccharides (e.g., mannitol) can transfer to the liquid phase. In these variations, the total polysaccharide composition and structure preferably does not substantially change (i.e., for all polysaccharides in the liquid and solid phases). However, the polysaccharides may (in other variations) change for one subset of the polysaccharides (e.g., liquid phase or solid phase but not both).

In a tenth illustrative example, the method of any of the first through ninth illustrative examples, wherein the alcohol comprises at least one of methanol, ethanol, or isopropyl alcohol.

In an eleventh illustrative example, the method of any of the first through tenth illustrative examples, wherein the alcohol is mixed with the algae sample as between about 25% and 80% w/w solution with water.

In a twelveth illustrative example, the method of any of the first through eleventh illustrative examples, wherein the algae sample comprises at least one of: Cymathaere triplicata, Laminaria ephemera, Laminaria longipes, Laminaria setchellii, Laminaria solidungula, Laminaria test, Laminaria yezoensis, integrifolia, Macrocystis pyrifera, Nereocystis luetkeana, Saccharina dentigera, Saccharina groenlandica, Saccharina latissima, Alaria marginata, or Saccharina sessilis.

In a thirteenth illustrative example, the method of any of the first through twelveth illustrative examples, wherein the algae sample is not dried prior to mixing with the alcohol.

In a fourteenth illustrative example, a mixture can comprise seaweed and at least about 8% by weight, relative to a wet weight of the seaweed, alcohol.

In a fifteenth illustrative example, the mixture of the fourteenth illustrative examples, wherein the seaweed comprises at least one of: Cymathaere triplicata, Laminaria ephemera, Laminaria longipes, Laminaria setchelli, Laminaria solidungula, Laminaria test, Laminaria yezoensis, integrifolia, Macrocystis pyrifera, Nereocystis luetkeana, Saccharina dentigera, Saccharina groenlandica, Saccharina latissima, Alaria marginata, or Saccharina sessilis.

In a sixteenth illustrative example, the mixture of any of the fourteenth through fifteenth illustrative examples, wherein a carbohydrate composition of the seaweed is between 30% and 80% on a wet basis.

In a seventeenth illustrative example, the mixture of the sixteenth illustrative examples, wherein the polysaccharide composition (and concentration) does not substantially change after a year (e.g., polysaccharides do not denature or depolymerize, polysaccharides remain in solid phase, polysaccharides transfer to liquid phase but do not degrade, total polysaccharide concentration between liquid phase and solid phase change by less than 5%, etc.). In some variations, one or more polysaccharides (e.g., mannitol) can transfer to the liquid phase. In these variations, the total polysaccharide composition and structure preferably does not substantially change (i.e., for all polysaccharides in the liquid and solid phases). However, the polysaccharides may (in other variations) change for one subset of the polysaccharides (e.g., liquid phase or solid phase but not both).

In an eighteenth illustrative example, the mixture of any of the fourteenth through seventeenth illustrative examples, wherein the alcohol comprises ethanol.

In a nineteenth illustrative example, the mixture of the eighteenth illustrative examples, wherein the ethanol comprises nondenatured ethanol.

In a twentieth illustrative example, the mixture of any of the fourteenth through nineteenth illustrative examples, wherein the seaweed comprises a length between about 500 μm and about 10 cm (e.g., 0.5-10 cm, 1-5 cm, 2-5 cm, 3-5 cm, 2-4 cm, 0.5-1 mm, 0.6-0.8 mm, 0.5-50 mm, etc.).

In a twenty-first illustrative example, the algae sample as formed by any of the first through thirteenth examples or the seaweed of any of the fourteenth through twentieth examples wherein a mass of algae or seaweed is between 10 kg and 50000 kg.

In a twenty-second illustrative example, a seaweed sample as formed by the method of any of the first through thirteenth illustrative examples.

In a twenty-third illustrative example, a method for forming a mixture of any of the fourteenth through twentieth illustrative examples.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the preceding system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

As a person of ordinary skill in the art will recognize, the reference to any specific clade, phylum, subphylum, class, and/or other taxonomic division can evolve over time and/or can depend on the taxonomic source. The use of such taxonomic distinctions herein are not limiting to any one classification system but are intended to illustrate potential systems and read with appropriate breadth. For instance, algae samples can include one or more of red seaweed, green seaweed, brown seaweed, yellow-green seaweed, and/or other suitable seaweeds or algaes.

As used herein, “substantially” or other words of approximation (e.g., “about,” “approximately,” etc.) can be within a predetermined error threshold or tolerance of a metric, component, or other reference (e.g., within 0.001%, 0.01%, 0.1%, 1%, 5%, 10%, 20%, 30% of a reference), or be otherwise interpreted.

We claim:

1. A method for preserving an algae sample comprising mixing the algae sample with an alcohol, wherein a concentration of the alcohol is at least about 8% by weight of the algae sample on a wet basis of the algae sample.

2. The method of claim 1, further comprising comminuting the algae sample prior to mixing the algae sample with the alcohol.

3. The method of claim 2, wherein after comminuting the algae sample, an average size of algae of the algae sample is between about 500 μm and 10 cm.

4. The method of claim 2, wherein comminuting the algae sample comprises cutting, grinding, high pressure homogenization, or shredding the algae sample.

5. The method of claim 1, further comprising storing the algae sample for a duration of time up to about one year.

6. The method of claim 5, wherein storing the algae sample comprises storing the algae sample at a temperature between −4° C. and 50° C.

7. The method of claim 5, wherein storing the algae sample comprises storing the algae sample in an unsealed container.

8. The method of claim 5, wherein after the duration of time, a carbohydrate composition of the algae sample does not substantially change.

9. The method of claim 8, wherein after the duration of time, a polysaccharide concentration of the algae sample does not substantially change.

10. The method of claim 1, wherein the alcohol comprises at least one of methanol, ethanol, or isopropyl alcohol.

11. The method of claim 1, wherein the alcohol is mixed with the algae sample as between about 25% and 80% w/w solution with water.

12. The method of claim 1, wherein the algae sample comprises at least one of:
Cymathaere triplicata, Laminaria ephemera, Laminaria longipes, Laminaria setchellii, Laminaria solidungula, Laminaria test, Laminaria yezoensis, integrifolia, Macrocystis pyrifera, Nereocystis luetkeana, Saccharina dentigera, Saccharina groenlandica, Saccharina latissima, Alaria marginata, or Saccharina sessilis.

13. The method of claim 1, wherein the algae sample is not dried prior to mixing with the alcohol.

14. A mixture comprising seaweed and at least about 8% by weight, relative to a wet weight of the seaweed, alcohol.

15. The mixture of claim 1, wherein the seaweed comprises at least one of:
Cymathaere triplicata, Laminaria ephemera, Laminaria longipes, Laminaria setchellii, Laminaria solidungula, Laminaria test, Laminaria yezoensis, integrifolia, Macrocystis pyrifera, Nereocystis luetkeana, Saccharina dentigera, Saccharina groenlandica, Saccharina latissima, Alaria marginata, or Saccharina sessilis.

16. The mixture of claim 1, wherein a carbohydrate composition of the seaweed is between 30% and 80% on a dry basis.

17. The mixture of claim 16, wherein the polysaccharide composition does not substantially change after a year.

18. The mixture of claim 1, wherein the alcohol comprises ethanol.

19. The mixture of claim 1, wherein a mass of the seaweed is between 10 kg and 50000 kg.

20. The mixture of claim 1, wherein the seaweed comprises a length between about 500 μm and about 10 cm.
